(19) United States
(12) Patent Application Publication
(10) Pub. No.: US 2004/0126901 A1
(43) Pub. Date: Jul. 1, 2004

(54) Clamped value beads

(76) Inventors: Lawrence M. Kauvar, San Francisco, CA (US); William D. Harriman, Alameda, CA (US)

Correspondence Address: MORRISON & FOERSTER LLP, 3811 VALLEY CENTRE DRIVE, SUITE 500, SAN DIEGO, CA, 92130-2332, US

(21) Appl. No.: 10/681,013

(22) Filed: Oct. 7, 2003

Related U.S. Application Data
(60) Provisional application No. 60/417,122, filed on Oct. 7, 2002.

(51) Int. Cl. G01N 33/543

(52) U.S. Cl. 436/523

(57) ABSTRACT

Compositions and methods which permit effective use of multiplexed particles of dimensions less than the resolution of the detector are useful in assessing the spatial pattern of targets or substructures within a specific environment.

### CROSS-REFERENCE TO RELATED APPLICATIONS

This application claims priority from U.S. Serial No. 60/417,122 filed 7 Oct. 2002 and incorporated herein by reference.

### TECHNICAL FIELD

The invention relates to assay methods including intracellular assays, using particulate labels smaller than the resolution of the imaging system. More particularly, it concerns enabling use of such small labels by allowing for assessment of the number of particulate (s) registered.

### BACKGROUND ART

U.S. patent application Ser. No. 09/146,984 filed Sep. 3, 1998 and 09/332,613 filed Jun. 14, 1999 and now published as PCT application PCT/US/99/19708, all of which are incorporated herein by reference, disclose particulate labels which are provided distinguishing characteristics using a combinatorial approach. As described in these documents, each different particulate label is provided a different “hue” by virtue of the presence of varied ratios of more than one signal-generating component. For example, one particle may contain a red wavelength emitting fluorophore and a blue wavelength emitting fluorophore in the ratio of 10:1, while a different label in the same collection is provided a red emitting fluorophore and a blue emitting fluorophore in the ratio of 1:4. The labels are thus distinguished by the different ratios of the fluorophores.

These labels are particularly useful when viewed under conditions where real-time analysis is possible using wide field microscopy as described in PCT application US 99/19086, also incorporated herein by reference. The availability of these techniques permits the viewer to ascertain not only the presence of these labels, as would be the case in more typical assays, but also to ascertain their position relative to other features of the environment.

In many instances, especially when it is desirable to view intracellular substructures, it is desirable to utilize such particulate labels that are smaller than the wavelength of visible light—i.e., smaller in diameter than about 400 nm, perhaps as small as 20 nm. As detecting such small particles using visible light inevitably requires a viewing area of at least 400 nm, the possibility/probability exists that more than a single particle will occupy this space. Therefore, it will be difficult to identify the relevant particulate label because the readout may be the result of the combination of two different labels.

For example, if the labels are distinguishable by virtue of the ratio of red to blue fluorophores coupled to their surface, the ratio obtained from detecting the smallest detection space containing these small particulate labels may be the result of a single label, or may be the result of a combination of emission from two different sources. A ratio of red:blue of 1:1 may result from the emission of a single particle bearing that ratio of fluorophores or may be the combination of two particles, one of which has a ratio of 10:1 and the other a ratio of 1:10 of red:blue.

In order to verify that the label is as detected, it is necessary, therefore, to verify that the signals are emitted only by a single particulate label. The present invention permits this verification.

### DISCLOSURE OF THE INVENTION

The invention relates to particulate labels that may be, but need not be, smaller in diameter than the resolution of the imaging system, and that are provided a “counter” in the form of a distinct color channel, other than the wavelengths used to create the distinctive hue, that is common to all the labels in a collection and that has a constant value per particle. Thus, for example, each particle in the collection may be provided with a known quantity, perhaps a single fluorescent object such as a quantum dot, that emits light of a particular wavelength. By measuring the intensity of emission at that wavelength upon detection of the particulate label, the presence of only a single particulate label in the detection space can be verified.

Thus, in one aspect, the invention is directed to a composition comprising a multiplicity of particulate different labels, wherein each different label comprises a particulate support to which is coupled at least one moiety which generates a visible signal characteristic of said label and further comprises a second signal generating moiety of standardized intensity, the wavelength of which signal is different from that or those which characterize(s) the label and wherein all of the particulate supports in the composition contain the identical second signal generating moiety. In a preferred embodiment, the different characterizing light emitting moieties are generated by at least two signal generating moieties wherein each of the moieties generates a signal different from that generated by the other(s) and wherein the magnitude of each said signals is varied among the different labels whereby each different label is thereby characterized by a different hue. Typically, the labels further contain a reagent which permits them to interact specifically with a desired target, such as an antibody or a nucleic acid.

In another aspect, the invention resides in providing a pattern of target substances which method comprises contacting an environment containing the target substances with the composition of the invention, preferably on a support suitable for viewing by wide field microscopy, and viewing the pattern generated by the distribution of the labels among the targets and further comprising assessing the number of particulate labels associated with the spatial viewing area of the detected label, and thereby verifying that the label is comprised of only a single particle.

In particular, the environment comprising the collection of targets may be an intracellular environment, or may constitute neural circuitry.

### MODES OF CARRYING OUT THE INVENTION

By virtue of the “clamped value” of a signal that serves as a counter according to the method of the invention, it is possible to utilize particulate labels which are smaller than the resolving power of the detector. This is advantageous, because the structural characteristics of environments to be analyzed may contain differentiating targets at distances within this range. Without the ability to verify that a particular label, as located and detected, is the result of the emission of a single particle, positive identification of the label is precluded.

As the “detection space” using visible light is inherently at least the dimension of the wavelength of light used for detection, it is possible that the detection space may contain more than one particle when such particles are indeed smaller than the detection space. By “detection space” is meant the area or volume represented by a signal which is detected, typically by means of microscopy techniques, such as confocal microscopy and wide field microscopy. The resolution of such microscopic techniques is ultimately limited by the wavelength of light that is detected. If the emitted light is at the blue end of the spectrum, the detection space has a diameter only of about 400 nm; at the red end of the spectrum, the minimal detection space is even larger—on the order of 750 μm. If a multiplicity of wavelengths is used, therefore, in order to ensure that all wavelengths are detected, a minimal detection volume or area will have a diameter of roughly 700-750 nm.

The methods of the invention are particularly convenient and helpful when the particles are smaller than the minimal detection of volume area; however, the compositions and methods of the invention may be employed even when this is not the case. One may arbitrarily choose a larger detection area than that mandated by the emitted radiation. Thus, the techniques of the invention apply to particulate labels of all sizes, not just those that are smaller than the minimal detection volume area.

The detection space is limited by the ultimate resolution of the detecting radiation—i.e., visible light, and not by the particular technique used for viewing the emitted light. Thus, the problems resolved by the compositions of the present invention are inherent in detecting particles by flow cytometry and by fiber optic detectors, to the extent that those devices cannot be miniaturized to the diameter of the particulate labels. However, as detection of spatial arrangements and relationships is a desirable outcome of supplying a multiplicity of labels to an environment with a multiplicity of targets, microscopy is clearly the preferred method of detecting the label composition.

In many instances, however, it is desirable to utilize particulate labels that are smaller in volume than represented by this diameter. In particular, for intracellular labeling, smaller particles are much better tolerated. It is desirable, in many instances, to utilize particles having diameters on the order of 20-200 nm. This means that the detection space can be occupied by more than one particle—e.g., if the particles are only 20 nm in diameter and the detection space is 700 μm in diameter, a multiplicity of such particles could crowd into this space.

As stated above, the particulate labels of the invention are used to establish the spatial relationships and patterns of various targets within an environment. By an “environment” is meant simply an area or volume of space which contains features that the viewer wishes to discern and distinguish. As used herein, these features will be referred to as “targets” as the compositions are designed so that each different particulate label in the composition contains a reagent which permits it to bind specifically to at least one of the several targets that may be contained in the environment. Thus, if the environment is the interior of a cell, the various targets may be the nucleus, the golgi apparatus, mitochondria, ribosomes, and the like. Within the environment of the nucleus, the targets may be the various appropriate DNA stretches characterizing portions of the chromosomes and/or the histones bound to them. Each different particulate label will comprise an appropriate binding reagent, for example, a nucleotide sequence that hybridizes specifically to a target gene, an aptamer that hybridizes to a particular stretch of DNA or which hybridizes to the golgi apparatus, an antibody that binds specifically to a receptor or a transduction mediator, and the like.

Thus, any convenient label may be used, depending on the partner to which the label it intended to be bound. Convenient labels include antibodies or fragments thereof, including recombinantly produced antibodies such as Fᵥ antibodies, fragments such as F_{ab} fragments, and the like. Ligands for particular receptors may be particularly useful. Peptides and peptidomimetics, aptamers, oligonucleotides designed to hybridize or bind through triplex formation, or any moiety which is the counterpart of a specific binding partner to be detected may be used as a label.

Similarly, the signal generating moieties, while typically fluorophores, may be any moieties that emit detectable signals and which can be supplied in gradient amounts. Various combinations of dyes, for example, might conveniently be used.

In a typical use for the compositions of the invention, the composition is applied to a desired environment, such as the interior of a cell and allowed to distribute according to the specifically binding labels. As each label associated with a different reagent is distinguishable from the other labels in the composition, the distribution of labels ultimately detected provides a pattern of the spatial distribution of the targets.

In viewing the distribution of targets, however, the smallest detection space available is that of the emitted signals. In the compositions of the invention, the signals are generated by light emitting substances or fluorophores. If only a limited number of targets were to be viewed within a set environment, so that only, for example, five different labels were required, it would be possible to assign each different label a different, distinguishable wavelength of emission and to count only those signals where the pre-established emission was viewed. However, the environments of greatest interest are much more complex and require a larger multiplicity of labels. This results in an overlap of signals unless there is some mechanism to assure that only a single label is detected.

The following is an illustration of the design of the labels in the composition which permits this verification as applied to multihued labels where the multiplicity of hues is established by varying the ratio of more than a single light emitting moiety or fluorophore. Three distinct particles may be prepared with three different reagents, R₁, R₂ and R₃, each of which binds to a different partner. Each particle has a different hue by virtue of the ratio of the characterizing fluorophores, F₁ and F₂, that emit 525 nm and 622 nm light, respectively. Each particle also has a “clamped value” of light emitted by F₃ at 425 nm of intensity A. Thus, an emission of this wavelength at intensity A will verify that only a single particle is being viewed. The characterizing emissions are of the same intensity in particle 1, but in particle 2, the 525 nm emitting moiety F₁ is of greater intensity than the 622 nm emitting moiety F₂; these intensities are reversed in particle 3. It will be evident that the same hue would be generated by a single particle with reagent 1 or by a combination of two particles, one with reagent 2 and the other with reagent 3. In order to establish the identity of the target/label, these possibilities must be distinguished.

This is particularly problematic in the circumstances wherein the particles are smaller than the wavelength of light. Typically, a target to be labeled is contained in a larger environment and will accumulate the appropriate labels. However, if the detection space within the target is larger than the individual particles, it will be desirable to distinguish detection spaces which contain only a single particle, from those which contain more than one particle. Where more than one particle is present, it cannot be stated with certainty that only the particles containing reagent R₁ have been bound. This can be ascertained by measuring the intensity of the 425 nm emission. If the intensity is A, only one particle is present; if the intensity is 2A, there are two particles present and if the intensity is 3A, there are three particles present and so forth.

Construction of the Particles

The material that constitutes the matrix of support for the visible light emitting fluorophores can be any particulate backbone. A multiplicity of such backbones is known, including latex, other polymeric supports such as Sephadex™ or diatomaceous earth, silica or glass particles, inert materials such a perfluorocarbons, and the like. Also useable as solid supports are viral particles, e.g.—poliovirus or a bacteriophage. Any material which exhibits particulate characteristics can be used.

In constructing the particles, a signal generating moiety that emits a single emission maximum, the narrower the better, is doped into the particles so that all of the particles in a particular composition have the same intensity of radiation at this wavelength. It is understood that modifications of this restriction might include supplying several different constant colors with clamped intensity per particle provided that number is relatively small compared to the variety of hues in the label. This variation is much more complex because the remaining signal generating moieties would have to be designed to permit discernment between the various clamped intensities; however, this is theoretically possible. Therefore, the requirement that all of the particles in the composition emit a specific wavelength at a clamped intensity includes this design possibility. It is also understood that the clamped intensity wavelength will represent an absorption maximum with a finite bandwidth for the emission spectrum. The wavelength restriction on the clamped intensity radiating moiety acknowledges the inherent shape of the emission spectrum and refers to the emission peak. It is desirable that this be as narrow as possible to avoid interference with the characterizing emitted signals which generate the hues of the various labels.

One approach employs a polymerization technique wherein a seed polymer comprises a fluorescent object such as a smaller particle with a single fluorophore that emits light at a predetermined intensity. One such particle is a quantum dot, these dots are bright enough so that one copy is sufficient; the clamped value is reliable and does not fade; and the emission bandwidth is narrow. See, for example, U.S. Pat. No. 6,423,551: “Organo Luminescent Semiconductor Nanocrystal Probes for Biological Applications and Process for Making and Using such Probes”, incorporated herein by reference.

However, alternative fluorophores already loaded in predetermined amounts by any manner could be used. For example the dyed seed particles could be sorted by flow cytometry to achieve a very narrow range of variation in emitted light intensity.

In the approach using a seed, such as a quantum dot, the particulate label can be obtained by initiating polymerization from the seed and either subsequently adding or binding the fluors which comprise the hue generating identifier or incorporating these in preset amounts into the polymerization reaction.

In the alternative, in an additional preferred approach, a virus particle may be used. Fluorophores may be intercalated into the nucleic acid characterizing the virus; in providing a variety of hues, all intercalation sites may be used. If, in order to create a different hue for which less than the total number of sites is occupied, a colorless dye can be used to complete the intercalation, thus assuring uniformity. To obtain the clamped value emitter, the emitter can be attached to a substance which binds to a unique site in the virus DNA, thus assuring that each packaged virus has a single package of emitting clamped value. Substances which will effect binding of the clamped value emitter to the virus nucleic acid include hybridizing oligomers, triplex forming nucleic acids, peptide nucleic acids, polyamides, antibodies, intercalators specific for particular sequences and the like.

In all of the foregoing cases, the particulate support is also provided a reagent which will bind to one specific desired target so that the identifying hue generating moieties will be characteristic of a particular interaction with target. Such binding reagents, e.g., antibodies, fragments of antibodies, peptidomimetics, aptamers, receptor-specific ligands, and the like can be coupled through standard linking technology to the particulate labels. In the specific case of particulate labels which are packaged viruses, the reagent is preferably a protein coupled to the coat protein for easy display. The clamped value DNA, reagent, and hue generating moieties may be added to the virus before or after packaging.

Typical protocols for preparing the clamped value beads include the following:

Generally available fluorescent dyes, including fluorescein (green), rhodamine (red), and DAPI (blue) are permeated into latex microspheres (e.g., 2 μm diameter beads manufactured by Interfacial Dynamics (Tualatin, Oreg.)) to prepare multihue beads, by swelling the bead in an organic solvent such as methanol or dimethyl formamide, then trapped inside by washing the beads in aqueous buffer. By adjusting the concentration of each dye in the permeating solution, beads are prepared having different ratios of the three dyes. Five intensity levels are clearly distinguishable, allowing 5×5×5=125 distinguishable types of bead, each of which is readily detectable with a 40× microscope objective lens and a high pixel density digital light detector (e.g., as provided by the DeltaVision microscope from Applied Precision (Seattle, Wash.)).

When imaging the same beads with a lower magnification lens and a light detector with fewer pixels, it is not feasible to resolve the individual beads, making it possible that one imaged bead-like object may in fact include contributions from two actual source beads. By clamping the concentration of one fluor, e.g., DAPI, in all beads to a fixed value, this source of error is eliminated since it becomes possible to determine whether or not an imaged bead-like object is arising from a single source bead. The cost of this error reduction is that only 5×5=25 bead types are distinguishable. The advantage is that an inexpensive imaging system can be used, which is useful for certain applications, such as multiplexed staining of Western blots (proteins separated by gel electrophoresis and then transferred to a membrane for staining and visualization).

Bacteriophage T7 particles are 50 nm in diameter, making them small enough to be suitable for use in staining intracellular antigens on fixed and permeabilized cells, with specificity for antigen achieved by conjugating an antibody to the phage or by engineering the phage production vector to include a fused antibody like domain. Alternatively, the antibody-phage conjugates are introduced into living cells via a cell permeating peptide such as TAT.

Soaking the phage in a solution of fluorescent dyes that intercalate between DNA residues results in fluorescent particles. By adjusting the ratio of commercially available dyes, particles can be prepared in multiple distinguishable types. For example, YOYO-1, POPO-1, CYBR Gold (all from Molecular Probes, Seattle Wash.) are dissolved at 10 μM and serially diluted by 2-fold increments before mixing with a suspension of phage for 12 hours. For each dye, approximately 6-8 distinguishable intensity levels are readable en masse. Individual particle variation is higher, and there is some interference among the dyes when mixed. Nonetheless, 10 hues are readily achievable with two dyes. Since these particles are too small to resolve by any generally available microscope, a method to count the number of actual source beads contributing to an imaged bead-like object is obtained by sacrificing one color channel to a clamped value.

Including a fourth distinguishable color in the particles increases the number of bead types that can be resolved. Since the excitation and emission spectra of conventional fluorescent dyes are broad, achieving four color channels requires sacrificing sensitivity, which limits the number of intensity levels that can be read. It is therefore advantageous to use a quantum dot as the clamped intensity fourth color, since these fluors have relatively sharp spectral bandwidths, which do not interfere with reading the conventional organic dye fluors. Alternatively, a chemiluminescent dye can be used to provide the fourth clamped color channel, using time resolved light detection to read the clamped color.

In either case, an advantageous method of assuring a fixed intensity of the clamped value fluor is to create a seed for bead polymerization that includes the clamp fluor. A single quantum dot is sufficient, given the high quantum yield of such fluors. For chemiluminescent or a fourth color of fluorescent dye, a dendrimer comprising a fixed number of copies of the dye is a suitable seed. Attaching a moiety that generates free radicals upon UV irradiation enables initiating bead polymerization from a solution of monomers. Each bead's growth, therefore, is initiated from a clamped value fluor. All of the resulting beads thus have equivalent intensity for the clamped value color.

Applications

The labeled compositions of the invention can be used for a wide variety of purposes where it is desired to ascertain the pattern of targets in an environment. Mapping of intracellular features is a particularly useful application. The small particle size of the members of the compositions of the invention is of particular advantage in intracellular labeling. Methods to introduce the composition of labels into cells are well known, including coupling the labels to a carrier protein such as TAT from HIV, the Drosophila ANTP peptide, or polymers which are disclosed in WO 02/10201; PCT/US01/23406: “Peptide-Mediated Delivery of Molecules Into Cells” assigned to Active Motif, Inc. Alternatively, the composition may be introduced into the cells by receptor mediated uptake into endosomes from which they are then released by subcellular trafficking signals. Use of selected peptides that bind to the heparin or chondroitin receptor, causing efficient uptake and subsequent release into either the cytoplasm or nucleus depending on the peptide are disclosed in Diatos application WO 01/64738: “Amino Acid Sequences Facilitating Penetration of A Substance of Interest Into Cells and/or Cell Nuclei.” It has been demonstrated previously that antibodies can be introduced into cells to immunoreact with antigens that reside intracellularly.

In another application, the particulate labels can be used as tracers for retrograde or anterograde labeling of neurons. Nanosphere delivery to subpopulations of neurons is described by Madison R., et al., Brain. Res. (1990) 522:90-98. By applying beads in a range of hues at one end, the functional anatomy is more readily visualized than is possible from a composite of animals each labeled sparsely with a single color of fluorescent particle.

Another application is illustrated on a macroscopic level, as the absolute size of the particle is not the key to utility of the present invention, but the ratio of particle size to resolution of the imaging system. The method of the invention may be applied to count cows grazing in a pasture, using generally available satellite imaging with a resolution of 6-10 feet. Labels emit radio waves of different frequencies, at different intensities, in lieu of the emitted light described above. By this means, all of the cattle belonging to each rancher can be identified by a rancher-specific signal frequency, and a grazing fee can then be assessed based on the number of each rancher's cows in a given pasture, which requires determination that individual cows are being counted. The expense of building and maintaining fences to restrict cows from one rancher's herd from grazing on another rancher's pasture is thereby rendered unnecessary. Such a system of electronic branding is relatively inexpensive to implement as compared to assigning each cow a unique electronic brand.

1. A composition comprising a multiplicity of particulate different labels, wherein each different label comprises a particulate support to which is coupled at least a first signal-generating moiety which generates a visible signal that characterizes said label, and
a second signal generating moiety of standardized intensity,
wherein the wavelength of the signal generated by said second signal-generating moiety is different from that which characterizes the label and
wherein all of the particulate supports in the composition generate the same signal from the second signal generating moiety and wherein said labels further contain a reagent which permits them to interact specifically with a desired target.

2. The composition of claim 1, wherein said characterizing signal generating moieties comprise at least two signal generating moieties wherein each of the moieties generates a signal different from that generated by the other(s) and wherein the magnitude of each said signals is varied among the different labels whereby each different label is thereby characterized by a different hue.

3. A method to provide a pattern of target substances which method comprises contacting an environment containing the target substances with the composition of claim 1 and viewing the pattern generated by the distribution of the labels among the targets, and
assessing the number of particulate labels associated with the detection space of any detected label.

4. The method of claim 3, wherein said environment is displayed on a support suitable for viewing by microscopy,

5. The method of claim 4, wherein the environment comprises the collection of targets is an intracellular environment.

6. The method of claim 4, wherein the environment comprises the collection of targets constitutes neural circuitry.
